(19) United States
(12) Patent Application Publication
(10) Pub. No.: US 2025/0012937 A1
(43) Pub. Date: Jan. 9, 2025

(54) ANTI-SCATTER GRID MISPLACMENT AND FOCAL POINT SOURCE OFFSET DETERMINATION FROM SHADOW MEASUREMENTS FOR A COMPUTED TOMOGRAPHY SYSTEM

(71) Applicant: CANON MEDICAL SYSTEMS CORPORATION, Tochigi (JP)

(72) Inventors: Xiaohui ZHAN, Vernon Hills, IL (US); Ruoqiao ZHANG, Vernon Hills, IL (US); Ilmar HEIN, Vernon Hills, IL (US)

(73) Assignee: CANON MEDICAL SYSTEMS CORPORATION, Tochigi (JP)

(21) Appl. No.: 18/348,479

(22) Filed: Jul. 7, 2023

(51) Int. Cl. G01T 7/00; G01T 1/29; G21K 1/02

(52) U.S. Cl. CPC G01T 7/005; G01T 1/2928; G01T 1/2985; G21K 1/025

(57) ABSTRACT

A method of determining a position offset includes receiving a first detection result from a first pixel of a plurality of pixels of a radiation detector, wherein the plurality of pixels are disposed on an incident side of the radiation detector on which an anti-scatter grid (ASG) is arranged, the plurality of pixels being aligned in at least a channel direction; receiving a second detection result from a second pixel of the plurality of pixels, wherein a septa of the ASG is arranged over a portion of the first pixel but is not arranged over any portion of the second pixel as viewed from the incident side of the radiation detector; estimating a positional offset of the septa of the ASG, based on the first detection result and the second detection result.

### BACKGROUND

### Field

The disclosure relates to a radiation detection apparatus used in a photon-counting computed tomography system.

### Description of the Related Art

For a typical scintillator detector-based computed tomography (CT) imaging system, the X-ray tube emits a certain number of photons during an exposure to the scanning object, and a detector array on the other side of the scanning object measures the transmitted photons, and then the measurement is normalized to an air scan at the same scan setting without the scanning object to estimate the attenuation of the path length. Therefore, the air scan and the object scan take place at different times, so any variation in the incident X-ray beam in the time domain needs to be calibrated for accurate measurement that leads to good image quality.

However, other than the X-ray tube flux change, the X-ray source position may also drift, depending on the tube type, over time due to the internal electrical steering variation and anode thermal expansion, as well as other design tolerances. Such positional variation usually would cause a random anti-scatter-grid (ASG) shadow profile change on the individual detector pixels and changing the measured intensity from time to time. Such X-ray source positional variation combined with non-ideal ASG angular alignment can cause different intensity drifts across the detector pixels, and result in ring artifacts in the reconstructed image. On the other hand, the ASG might also experience certain deformation due to high rotation speed, and cause positional and rotational speed-dependent intensity variation across the pixels.

For a semiconductor (CdTe/CZT)-based photon counting CT (PCCT), the typical detector array design usually has a much smaller pixel size compared to the conventional CT detector, due to the trade-off between charging sharing effect and the pulse pile up effect to achieve the best energy resolving performance. Typically, the pixel pitch is chosen between 250 μm and 500 μm in one dimension, compared to ˜1 mm for the conventional pixel pitch. Thus, the conventional detector pixel area is usually equivalent to a N×N group of sub-pixels in PCCT, where N can be between 2 to 4. To maintain high dose efficiency, the ASG design usually still remains in the same pitch/spacing as a conventional system pixel distribution.

One important application for the PCCT is spectral imaging. To achieve good performance, accurate X-ray source position and ASG alignment is needed to solve the problem of measuring the viability of a detector module of the radiation detector. Current technology does not possess a means of physically measuring the misalignment of the ASG or the X-ray source position offset. Therefore, a new method of radiation detector design is also needed for monitoring/calibration purposes without the use of external measuring tools.

For a small, pixelated PCD design, the ASG plates usually keep the same spacing as the conventional CT design. A 3×3 sub-pixel scheme is used in an exemplary embodiment, for which each sub-pixel is ˜⅓ of the conventional detector pixel size. In this design, the ASG shadow only affects sub-pixels 1 and 3 in each group, and the middle sub-pixel is not affected by those effects, as previously described. Therefore, even with a perfect ASG plate alignment, the sub-pixel readout would always have normalization error across the detector along the X-ray source movement direction, and this is a random correction factor that no existing apparatus can resolve. This would generate ring artifacts in the high-resolution images that use the sub-pixel level readout for image reconstruction.

In practice, the ASG plates almost always have certain mechanical tolerances for both positional accuracy and angular accuracy. Therefore, the combined readout (e.g., 3×3 summing mode) would also encounter the same problem as described above and generate a ring artifact in the standard resolution images that use the combined pixel readout for reconstruction, when this effect is significant enough.

### SUMMARY

In one exemplary embodiment, there is provided an apparatus, comprising: (1) radiation detector including a plurality of pixels; (2) an anti-scatter grid (ASG) arranged on an incident side of the radiation detector, wherein a septa of the ASG is arranged over a portion of a first pixel of the plurality of pixels, but is not arranged over any portion of a second pixel of the plurality of pixels, as viewed from the incident side of the radiation detector; and (3) processing circuitry configured to receive a first detection result from the first pixel; receive a second detection result from the second pixel; estimate a positional offset of the septa of the ASG, based on the first detection result and the second detection result.

In another exemplary embodiment, there is provided an apparatus for detecting misalignment of an anti-scattering grid (ASG) offset detection for a radiation detector apparatus, the apparatus comprising: (1) a radiation detector including a plurality of pixels, the plurality of pixels being arranged in a plurality of modules; and (2) processing circuitry configured to acquire first counts obtained from a first air scan with an anti-scatter grid (ASG) having a plurality of septa arranged on an incident side of the radiation detector; acquire second counts obtained from a second air scan without the ASG arranged on the radiation detector; calculate third counts by normalizing the first counts based on the second counts; estimate positional offsets of the plurality of septa of the ASG based on the calculated third counts; determine whether any particular module of the plurality of modules is faulty based on the estimated positional offsets of the plurality of septa.

In another exemplary embodiment, there is provided an apparatus for detecting a tilt angle of an X-ray source, comprising: (1) a radiation detector including a plurality of pixels, the plurality of pixels being arranged in a plurality of channels; (2) an anti-scatter grid (ASG) arranged on an incident side of the radiation detector, wherein, for each channel of the plurality of channels, as viewed from the incident side of the radiation detector, (1) a first septa of the ASG is arranged over a portion of a first pixel of the channel, but is not arranged over any portion of a second pixel of the channel that is adjacent to the first pixel, and (2) a second septa of the ASG is arranged over a portion of a third pixel of the channel that is adjacent to the second pixel, but is not arranged over any portion of the second pixel, the first, second, and third pixels forming the channel; and (3) processing circuitry configured to, for each channel of the plurality of channels, acquire a first detection result from the first pixel, a second detection result from the second pixel, and a third detection result from the third pixel, the detection results being obtained from an air scan with the ASG using the radiation source; estimate a right offset of the first septa, based on the first detection result and the second detection result; estimate a left offset of the second septa, based on the third detection result and the second detection result; generate a mean right offset by averaging the estimated right offset over each of the channels, and generate a mean left offset by averaging the estimated left offset over each of the plurality of channels; determine the tilt angle of the X-ray source based on the generated mean right offset and the generated mean left offset.

### BRIEF DESCRIPTION OF THE DRAWINGS

The application will be better understood in light of the description which is given in a non-limiting manner, accompanied by the attached drawings in which:

FIG. 1 shows a schematic of a radiation detector design comprising a plurality of microchannels, wherein an ASG is mounted on a plurality of pixels in accordance with an embodiment;

FIG. 2 shows a radiation detector design comprising a two-dimensional array of a plurality of detector module blades (DMB) in accordance with an embodiment;

FIG. 3A shows four examples of CZT submodule misalignment in accordance with an embodiment;

FIG. 3B shows an example ASG misalignment with respect to the radiation detector design in accordance with an embodiment;

FIG. 4 shows a schematic of ray tracing of X-ray photons generated by an X-ray source opposing the radiation detector, wherein the ray tracing of the X-ray photons shows a plurality of shadows cast on the plurality of pixels by the ASG in accordance with an embodiment;

FIG. 5 shows a flowchart of a method of identifying an ASG offset according to an embodiment;

FIGS. 6A-6F show examples of ASG misalignment in accordance with an embodiment;

FIG. 7A shows a schematic of an X-ray source misalignment opposing the radiation detector in accordance with an embodiment;

FIG. 7B shows a schematic of the X-ray source misalignment opposing the radiation detector with the ASG included in accordance with an embodiment;

FIG. 8 shows a flowchart of a method of identifying an X-ray source tilt angle in accordance with an embodiment;

FIG. 9A shows plots of a plurality of right ASG offsets and a plurality of left ASG offsets for a positive and a negative X-ray source tilt angle according to an embodiment;

FIGS. 9B and 9C show, respectively, a predictive tilt-versus-offset table generated for a positive rotation corresponding to the ASG offset right, and a predictive tilt-versus-offset table generated for a negative rotation corresponding to the ASG offset left according to an embodiment;

FIGS. 9D, 9E, and 9F illustrate an example of determining a tilt angle based on calculated offsets;

FIGS. 10A and 10B illustrate the geometry of the septa of the ASG for focal spot tilt;

FIG. 10C illustrates example geometry for ASG offset calculations;

FIG. 11 shows a block diagram that illustrates an example of a configuration of an X-ray CT apparatus according to an embodiment;

FIG. 12 shows an example computer system configured to implement the methods of the disclosed invention according to an embodiment; and

FIG. 13 shows another example computer system configured to implement the methods of the disclosed invention according to an embodiment.

### DETAILED DESCRIPTION

Reference throughout this specification to “one embodiment” or “an embodiment” means that a particular feature, structure, material, or characteristic described in connection with the embodiment is included in at least one embodiment of the application, but do not denote that they are present in every embodiment.

Thus, the appearances of the phrases “in one embodiment” or “in an embodiment” in various places throughout this specification are not necessarily referring to the same embodiment of the application. Furthermore, the particular features, structures, materials, or characteristics may be combined in any suitable manner in one or more embodiments.

Aspects of this embodiment are directed to a radiation detector design for identifying ASG offset and X-ray source misalignment. The radiation detector design includes a radiation detector apparatus opposite an X-ray emitter comprised of a two-dimensional array of 38 DMB modules in one embodiment. Simulated offsets were generated and compared to the mean offset calculated by the method of the radiation detector design corresponding with a tilt angle in accordance with one embodiment.

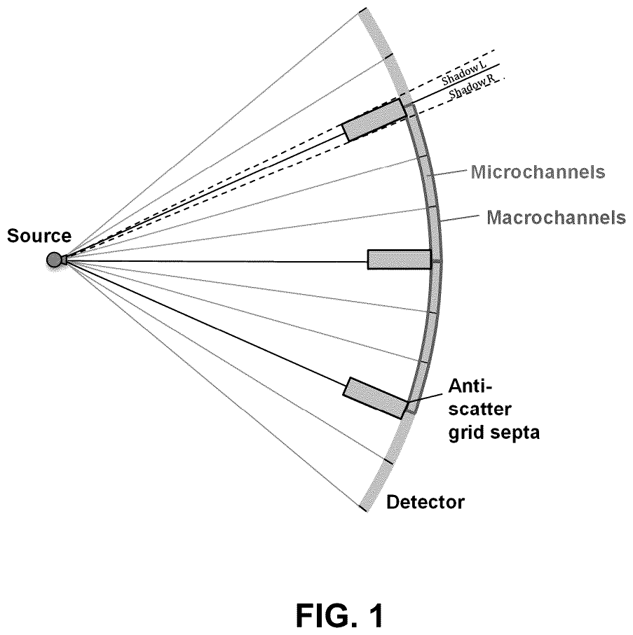

FIG. 1 illustrates an exemplary embodiment of a radiation detector, wherein an X-ray source is opposed by a curved detector surface, the detector surface including a plurality of macro channels. Each channel is comprised of three microchannels according to one embodiment. A septum of the ASG is disposed on the right edge and the left edge of each of the plurality of macro channels. Each of the microchannels has an equal pixel width according to one example.

The X-ray source emits photons and the microchannels are configured to absorb the emitted photons and convert the photons into an electric signal. The photons travel along a straight line such that the septa of the ASG can cast a shadow (e.g., a left shadow and a right shadow) on the adjacent microchannels, wherein the shadows cast by the septa will reduce the photon absorption by the detector.

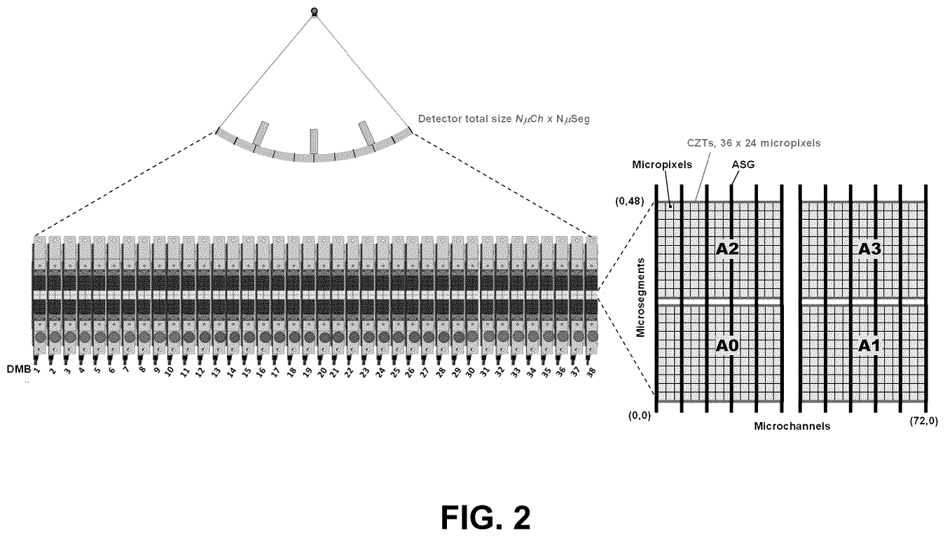

FIG. 2 illustrates a radiation detector apparatus comprising a plurality of DMB. In one embodiment the radiation detector apparatus includes 38 DMB each having detector pixels facing towards the X-ray source. In an aspect, each of the DMB can be further subdivided into four CZT submodules. Each of the CZT submodules can include, for example, a 36×24 array of micropixels in accordance with one embodiment.

In one embodiment, the micropixels in the row direction of each of the DMB are denoted as micro rows, the micropixels in the column direction of each of the DMB are denoted as micro channels. In one exemplary embodiment, the ASG are disposed bisecting every third microchannel. In this arrangement, the ASG septa cast no shadow on the central micropixel and filter out scattered X-ray photons emitted by the X-ray source, thus improving image quality and avoiding issues that would degrade the image, such as ring artifacts.

In another embodiment, each of the DMB comprises an array of 12 CZT submodules arranged in a 2×6 grid pattern, wherein the ASG are disposed on every third micropixel of each of the CZT. In this embodiment, each septum of the ASG comprises a lead strip separated by a low attenuating interspace material such as carbon fiber or aluminum running along in between every third micropixel.

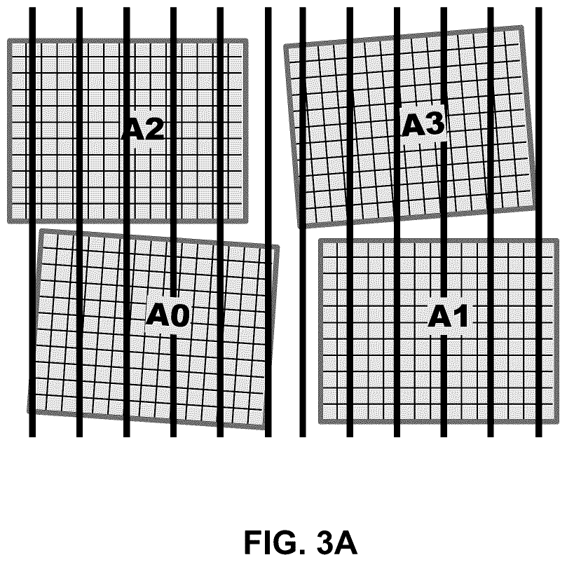

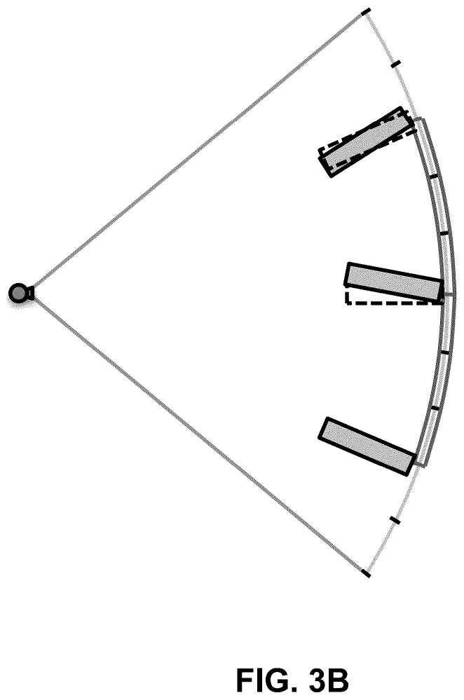

FIG. 3A illustrates an embodiment of a DMB misalignment. A misalignment of the DMB can comprise a rotation of a CZT submodule due to a misplacement of the CZT submodule during installation. In another embodiment, the misalignment of the CZT submodules can be due to a deformation due to high rotation speed of the apparatus, causing a positional and a rotational speed dependent intensity variation across the pixels. In another embodiment the CZT submodules are positioned on a substrate in a 2D grid and the micropixels possessing a photoelectric conversion unit, which is arranged on a scintillator.

FIG. 3B illustrates an example of an ASG misalignment occurring on a curved surface of the radiation detector apparatus, wherein the X-ray source opposite the ASG emits X-ray photons in a straight line. In this example, the dashed lines represent an ideal position of each septum of the ASG, wherein an offset of an ASG septum can comprise a left rotation of the septum, a right rotation of the septum, or a translational shift of the septum (or combinations thereof).

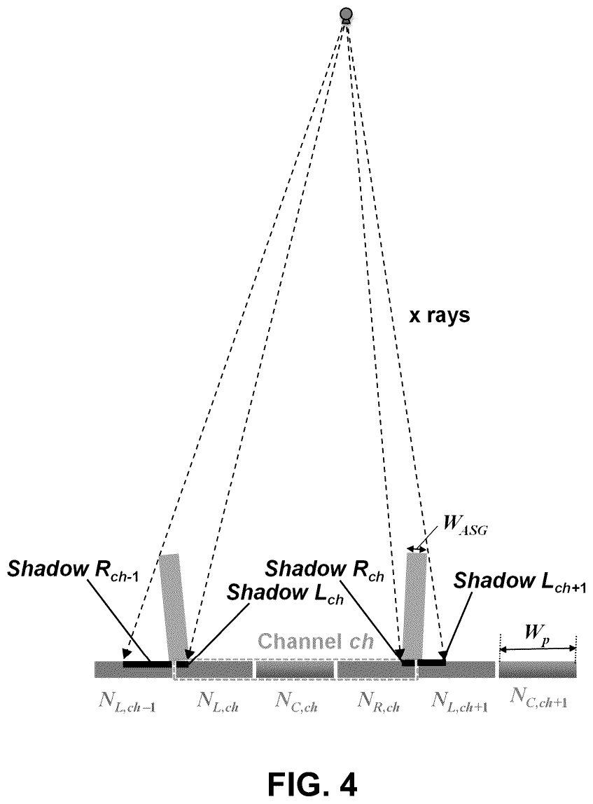

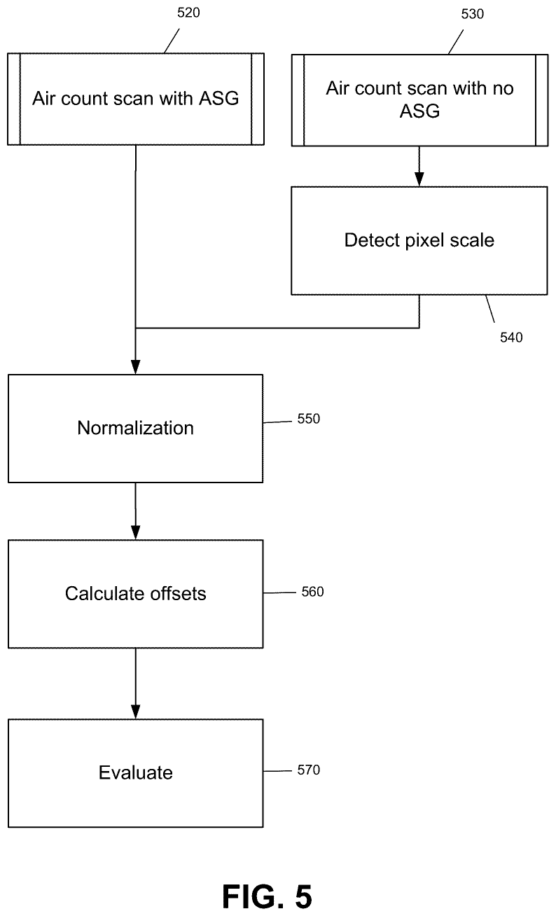

While a certain amount of ASG misalignment can be tolerated by most PCCT systems, ASG misalignment exceeding a mechanical tolerance of the PCCT system can lead to a degradation of the final produced image. The present disclosure addresses the limitation of physically measuring the misalignments of both the detector elements and the ASG via the methodology described below with respect to FIG. 4 and FIG. 5.

FIG. 4 illustrates an example misalignment of the ASG, wherein the ASG casts a shadow across some of the micropixels of a channel of three micropixels. Assuming that both the ASG and the micropixels are accurately positioned and aligned, the ASG's shadow will be consistent for all ASG septa positions and corresponding micropixels. During an air scan, the measured count for a specific micropixel will be directly proportional to the non-shadowed region of that micropixel. As shown in FIG. 4, the central microchannel of the channel lacks both septa and shadow, resulting in a full count.

However, if there is some misalignment between the ASG and the micropixels, as shown in FIG. 4, the counts and shadow lengths for the left and right micropixels of a channel will deviate from the ideal scenario. Equations 1˜4 below are used to calculate the left and right shadow lengths and corresponding left and right ASG offsets, based on the count at each micropixel, the ASG septa width, and the micropixel width:

$$Shadow {R}_{ch} = {W}_{p} ( 1 - \frac{{N}_{R , ch}}{{N}_{C , ch}} ) \qquad ( Eq . 1 )$$

$$Shadow {L}_{ch} = {W}_{p} ( 1 - \frac{{N}_{L , ch}}{{N}_{C , ch}} ) \qquad ( Eq . 2 )$$

$$ASG Offset R = Shadow Rch - ( {W}_{ASG} / 2 ) \qquad ( Eq . 3 )$$

$$ASG Offset L = Shadow Lch - ( {W}_{ASG} / 2 ) \qquad ( Eq . 4 )$$

wherein Wₚ is the micropixel width, N_{R,ch} is an air count for channel right microchannel, N_{L,ch} is an air count for channel left microchannel, N_{C,ch} is an air count for channel center microchannel, ch is a channel identifier, and W_{ASG} is the ASG septa width.

According to one embodiment, using Equations 1 and 2, processing circuitry is configured to calculate a shadow amount for the right and left microchannels of a given channel (Shadow R_{ch} and Shadow L_{ch}), based on the air counts measured for the microchannel and Wₚ. Next, using Equations 3 and 4, the processing circuitry is configured to calculate the left and right ASG offsets based on the calculated left and right shadow amounts and the width of the corresponding ASG septa W_{ASG}.

FIG. 5 illustrates a flow chart of a method employing the calculations of Equations 1-4.

In step 520, an air scan is performed with the ASG to obtain first air scan counts at each microchannel.

In step 530, an air scan is performed without the ASG to obtain second air scan counts at each microchannel.

In step 540, the second air scan counts are divided by the corresponding pixel widths to account for pixels of different sizes.

In step 550, the first air scan counts are normalized by the second air scan counts to obtain the values N_{R,ch}, N_{L,ch}, N_{C,ch} used in Equations 1 and 2, for example.

In step 560, Equations 3 and 4 set forth above are used to calculate the left and right offsets for the septa of the ASG for each channel.

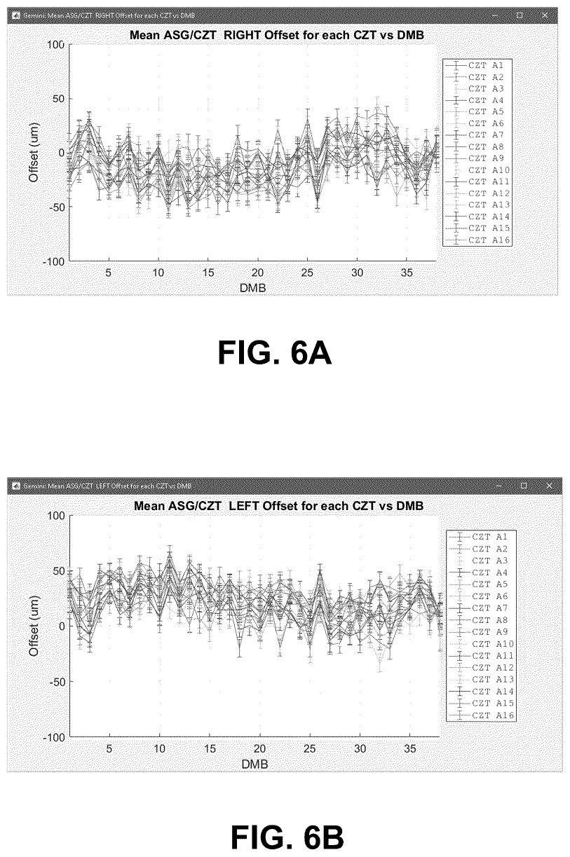

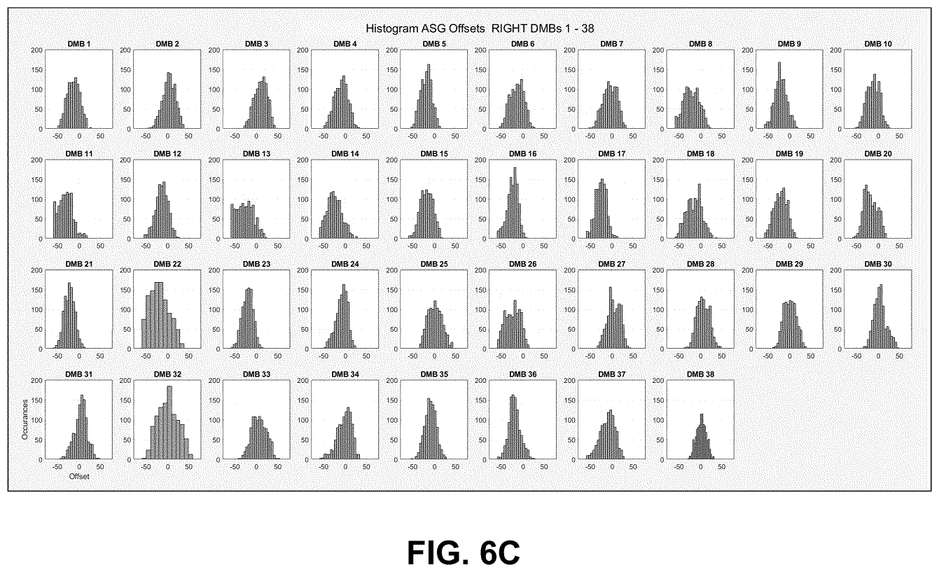

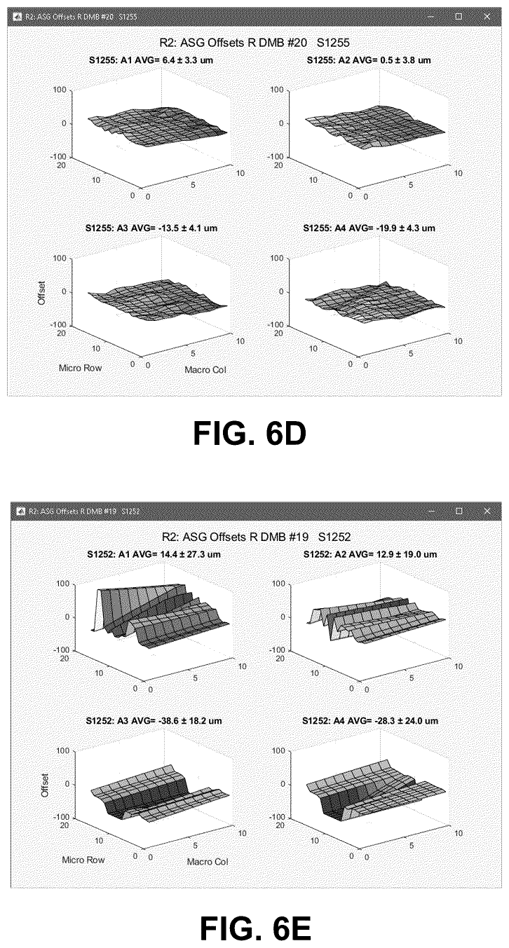

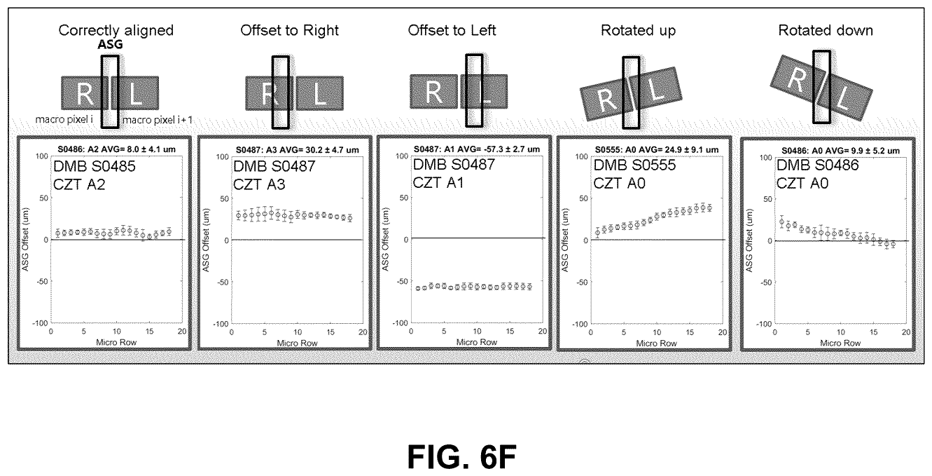

In step 570, the offset data calculated for each channel can be evaluated and displayed to determine whether a particular DMB module is faulty, for example, as shown in FIGS. 6A-6F, discussed below.

FIGS. 6A and 6B illustrate example average right and left offsets, respectively, for a system comprising 16 submodules per DMB. The mean offset value for each submodule in a given DMB is plotted. The mean offset is considered acceptable if it falls within a range of plus or minus 50 micrometers. However, if a particular DMB displays a very large offset outside the predetermined tolerance of the radiation detector system, it would indicate that the DMB is defective and requires replacement.

As shown in FIG. 6C, the results of the mean ASG offsets can be plotted as histograms for each DMB according to another embodiment. Here, the histograms for DMBs 11 and 13 appear to be abnormal, indicating that the DMB quality should be further investigated.

FIGS. 6D and 6E illustrate the offsets plotted in a 3D format for each submodule in a DMB. The four plots in FIG. 6D show four submodules in a good DMB, in which the surfaces are relatively flat and uniform with low offset values. A bad DMB plot is shown in the four plots in FIG. 6E, wherein the surface is irregular and discontinuous with high offset values

FIG. 6F illustrates a multitude of possible types of misalignments of the ASG and the corresponding offset profiles. To determine the rotational misalignment and shift misalignment of the ASG offsets, one can plot the offsets along the micro row (microsegment) direction for a specific submodule. The plots in FIG. 6F depict the various offset patterns for normal, left- and right-shifted, and up- and down-rotated ASG placements.

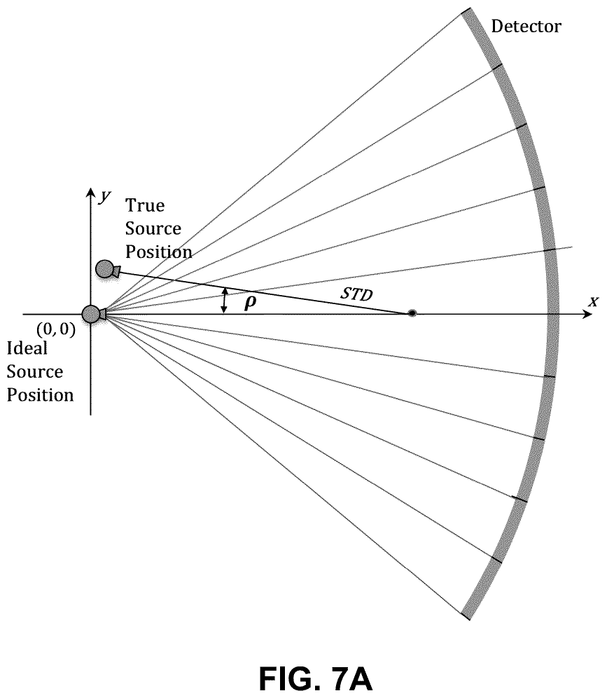

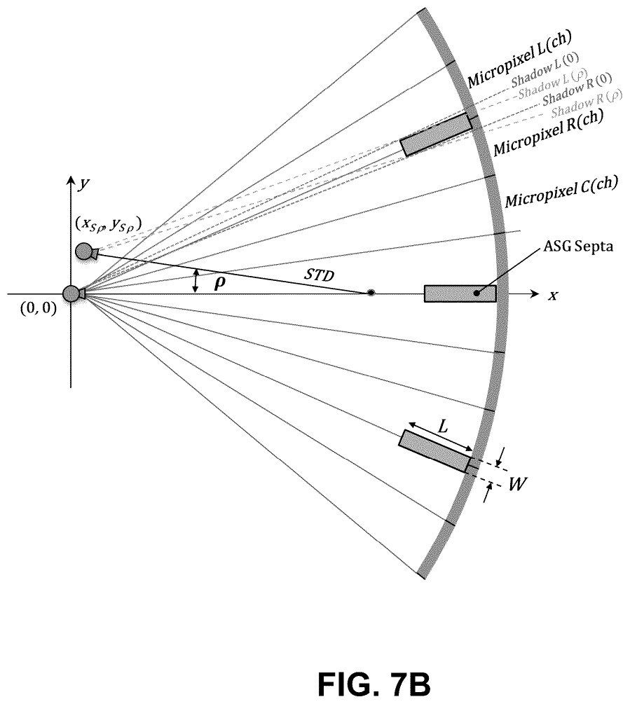

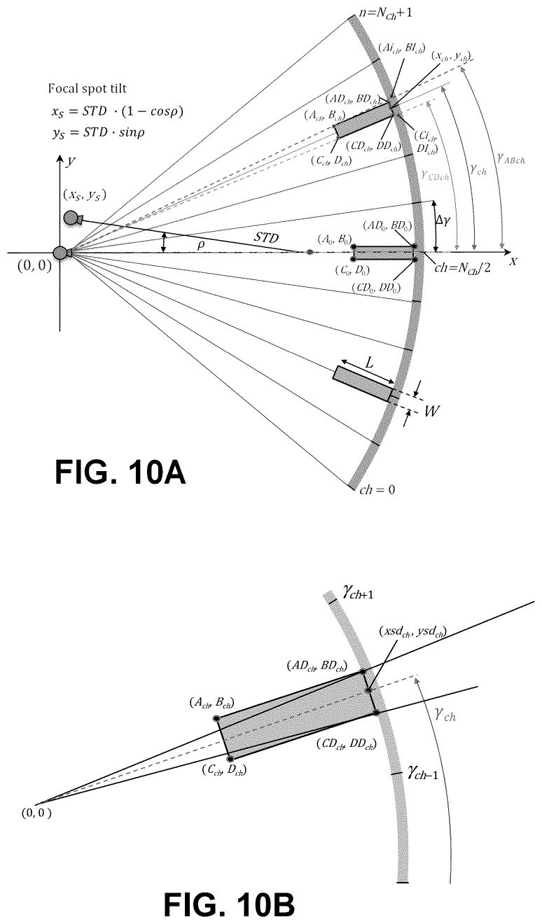

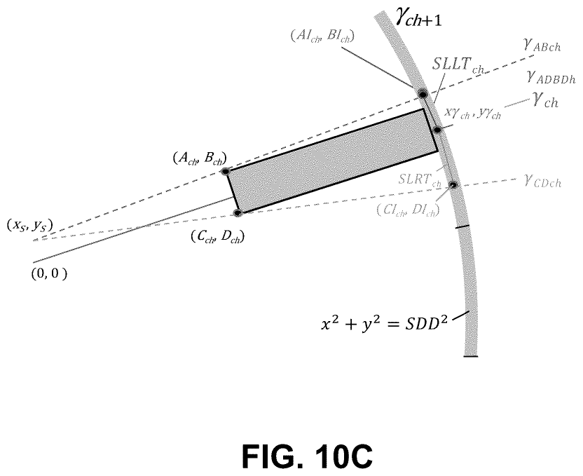

In a second embodiment, as shown in FIG. 7A, the X-ray source in a PCCT system can be tilted by a source tilt angle ρ with respect to the isocenter at a distance STD, and this offset of the X-ray source can cause shadows to be cast by the ASG on the radiation detector apparatus. These shadows can affect the quality of the images produced by the radiation detector apparatus. As shown in FIG. 10A, the X-ray source tilt may be defined by defining coordinates x_{S}=STD. (1−cos(ρ)) and y_{S}=STD·sin(ρ) according to one embodiment, wherein x_{S} is the x-coordinate of the X-ray source location in a 2D space, y_{S} is the y-coordinate of the X-ray source location in the 2D space, and STD is the distance from the isocenter to the X-ray source location.

FIG. 7B illustrates the X-ray source tilt of FIG. 7A within the installation of an ASG on the radiation detector apparatus in this embodiment. Similar to the embodiment discussed above with respect to FIGS. 1 and 4, the PCCT imaging system in this embodiment utilizes ASG septa located between every three microchannels disposed on the radiation detector apparatus. When there is no X-ray source tilt (ρ=0), the ASG septa cast shadows of length Shadow L(0) and Shadow R(0) on a left micropixel and a right micropixel, respectively, on the detector. However, when the X-ray source is tilted by p, the lengths of the shadows, Shadow L(ρ) and Shadow R(ρ), are altered depending on ρ. These shadow lengths can be calculated from the micropixel air counts from an air scan, and are directly proportional to the air counts from the air scan. Equations 1˜4 discussed above can be used to calculate the Shadow L(ρ), the Shadow R(ρ), the left ASG offset, and the right ASG offset.

In one embodiment, pre-calculated values of the left ASG offset and the right ASG offset for an ideal PCCT imaging system are calculated via simulation for a corresponding range of input p values. A lookup table is thus generated and can be used to look up a value of p when a measured left ASG offset and a measured right ASG offset are provided. This approach offers a solution for identifying the tilt angle ρ via the values of the left ASG offset and the right ASG offset, and provides a useful tool for PCCT imaging.

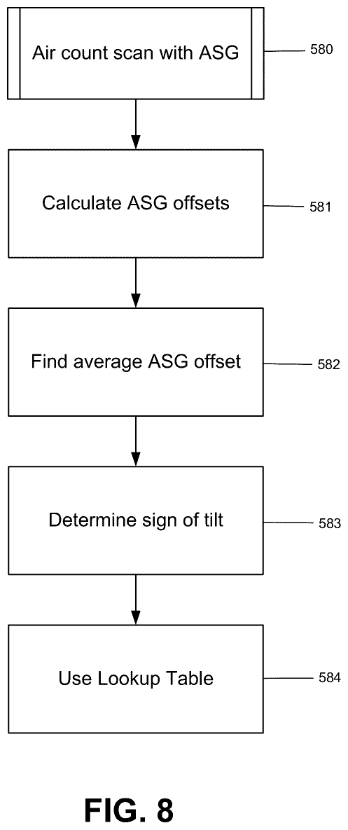

FIG. 8 is a flowchart of a method of calculating the source tilt angle according to this embodiment. As noted above, a lookup table (or multiple tables depending on tilt angle sign, as discussed below) associating an input mean ASG offset and output tilt angle ρ is precalculated via simulation.

In step 580, an air scan is performed with the ASG in place to obtain air scan counts at each microchannel, as described above.

In step 581, the left and right offsets are computed using Equations 1-4, as discussed above.

In step 582, the mean right offsets and the mean left offsets are calculated for each channel.

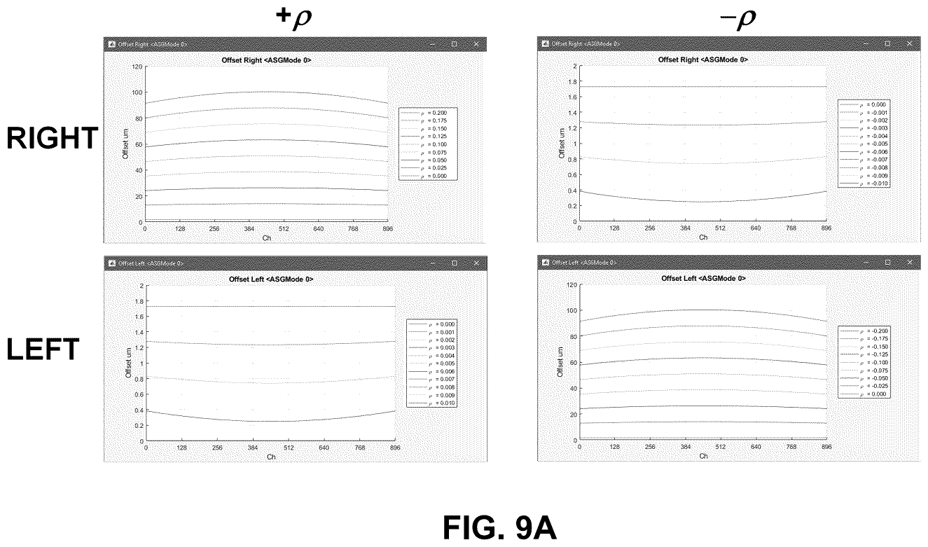

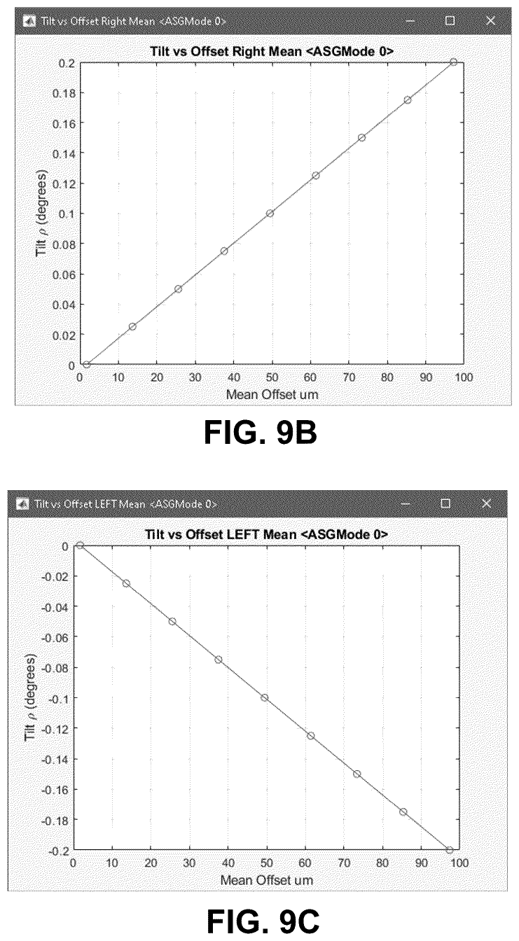

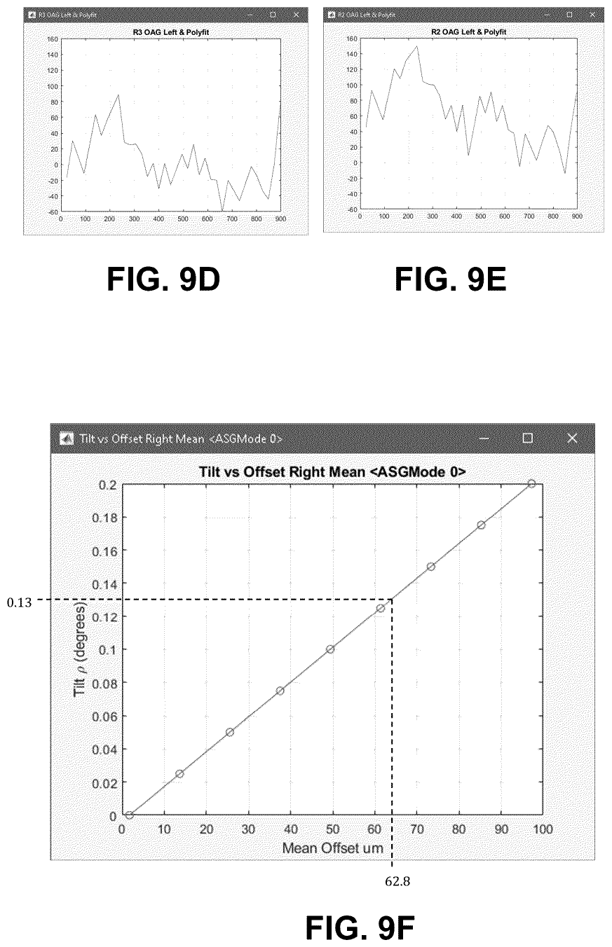

In step 583, the sign of the tilt (positive or negative) is determined based on the mean left and right offsets. In one embodiment, the sign of the tilt (positive or negative) is first determined by comparing a graph of the left and right offsets over all of the channels to the corresponding graphs shown in FIG. 9A. In particular, the shape of the left and right offset graphs are different depending on whether the tilt is positive or negative, as shown in FIG. 9A. For a positive tilt angle, the right ASG offsets are much greater than the left ASG offsets. For a negative tilt angle, the reverse is true, and the right ASG offsets are much smaller in magnitude than the left ASG offsets.

In step 584, the predetermined lookup tables are used to determine the values of the tilt angle. Alternatively, a single combined lookup table or other database structure can be used. For a positive tilt angle determined in step 583, i.e., if the mean ASG right offset is greater than the mean ASG left offset, the mean ASG right offset table is used, as shown for example in FIG. 9B. For a negative tilt angle determined in step 583, i.e., if the mean ASG right offset is less than the mean ASG left offset, the mean ASG left offset table is used, as shown in FIG. 9B, for example.

In one example, FIGS. 9D and 9E show ASG Offset data for two datasets R3 and R2. R3 had a presumed (not known exactly) ρ=0.00° and R2 with ρ=0.15°. The shape of the real ASG offsets is very noisy due to other factors such as individual detector CZT micropixel misplacements. However, it is not the shape, but the mean ASG offset that is primarily affected by the tilt, so the tables are valid. The measured mean ASG Offset for R3 was 2.77, and for R2 was 62.76. From the table, we calculated ρ=0.00° for R3, and calculated ρ=0.13 for R2, as shown.

FIG. 10A illustrates an example of the geometry of a single ASG septa disposed on the radiation detector of FIG. 1 with a focal spot tilt according to an embodiment wherein the ASG septa are fixed to the boundary between the microchannels of FIG. 1. Here, the coordinates of the septum at the detector center is given by:

$$\begin{matrix}{A}_{0} = SDD - L & {AD}_{0} = SDD \\ {B}_{0} = \frac{w}{2} & {BD}_{0} = \frac{w}{2} \\ {C}_{0} = SDD - L & {CD}_{0} = SDD \\ {D}_{0} = \frac{- w}{2} & {DD}_{0} = \frac{- w}{2}\end{matrix}$$

FIG. 10B illustrates and example of the geometry for the septa positions in one embodiment. Here, the ASG septa coordinates are given by:

$$\begin{matrix}{xsd}_{ch} = SDD \cdot cos ( {γ}_{ch} ) & \\ {ysd}_{ch} = SDD \cdot sin ( {γ}_{ch} ) & \\ {A}_{ch} = {R}_{AB} \cdot cos ( {q}_{AB} + {γ}_{ch} ) & {AD}_{ch} = {R}_{ADBD} \cdot cos ( {q}_{ADBD} + {γ}_{ch} ) \\ {B}_{ch} = {R}_{AB} \cdot sin ( {q}_{AB} + {γ}_{ch} ) & {BD}_{ch} = {R}_{ADBD} \cdot sin ( {q}_{ADBD} + {γ}_{ch} ) \\ {C}_{ch} = {R}_{CD} \cdot cos ( {q}_{CD} + {γ}_{ch} ) & {CD}_{ch} = {R}_{CDDD} \cdot cos ( {q}_{CDDD} + {γ}_{ch} ) \\ {D}_{ch} = {R}_{CD} \cdot sin ( {q}_{CD} + {γ}_{ch} ) & {DD}_{ch} = {R}_{CDDD} \cdot sin ( {q}_{CDDD} + {γ}_{c h} )\end{matrix}$$

$$where$$

$$\begin{matrix}{γ}_{ch} = ( ch - \frac{{N}_{Ch}}{2} ) \cdot Δγ & ( 0 < ch < {N}_{Ch} + 1 ) \\ {γ}_{ADBDch} = {γ}_{ch} + \frac{Δγ}{2} & {γ}_{CDDDch} = {γ}_{ch} - \frac{Δγ}{2} \\ {q}_{AB} = {tan}^{- 1} ( \frac{{B}_{0}}{{A}_{0}} ) & {q}_{ADBD} = {tan}^{- 1} ( \frac{{BD}_{0}}{{AD}_{0}} ) \\ {q}_{CD} = {tan}^{- 1} ( \frac{{D}_{0}}{{C}_{0}} ) & {q}_{CDDD} = {tan}^{- 1} ( \frac{{DD}_{0}}{{CD}_{0}} ) \\ {R}_{AB} = \sqrt{{A}_{0}^{2} + {B}_{0}^{2}} & {R}_{ADBD} = \sqrt{{AD}_{0}^{2} + {BD}_{0}^{2}} \\ {R}_{CD} = \sqrt{{C}_{0}^{2} + {D}_{0}^{2}} & {R}_{CDDD} = \sqrt{{CD}_{0}^{2} + {DD}_{0}^{2}}\end{matrix} .$$

FIG. 10C illustrates the geometry for performing ASG offset calculations according to one embodiment. In particular, the left and right offsets can be given by:

$$ASG\_Offset \_Leftch = {SLLT}_{ch} - \frac{W}{2}$$

$$ASG\_Offset \_Rightch = {SLRT}_{ch} - \frac{W}{2}$$

wherein:

$${SLLT}_{ch} = \sqrt{{( {AI}_{ch} - {x}_{ch} )}^{2} + {( {BI}_{ch} - {y}_{ch} )}^{2}}$$

$${SLRT}_{ch} = \sqrt{{( {CI}_{ch} - {x}_{ch} )}^{2} + {( {DI}_{ch} - {y}_{ch} )}^{2}}$$

Further, the front septa ray-to-detector intersection points and angles are given by:

$$\begin{matrix}X , Y = A , B ; C , D ; M = AB ; CD ; \\ XI , YI = AI , BI ; CI , DI ; \\ {γ}_{Mch} = {tan}^{- 1} \frac{{YI}_{ch}}{{XI}_{ch}} \\ {YI}_{ch} = {m}_{Mch} \cdot {XI}_{ch} + {b}_{Mch} \\ {AI}_{ch} = \frac{- {F}_{Mch} \pm \sqrt{{F}_{Mn}^{2} - 4 {E}_{Mch} {G}_{Mch}}}{2 {E}_{Mch}} \\ {E}_{Mch} = 1 + {m}_{Mch}^{2} \\ {F}_{Mch} = 2 \cdot {m}_{Mch} \cdot {b}_{Mch} \\ {G}_{Mch} = {b}_{Mch} - {SDD}^{2}\end{matrix}$$

The source-septa corner array equations are:

$$\begin{matrix}{y}_{Mch} = {m}_{Mch} \cdot x + {b}_{Mch} \\ {m}_{Mch} = \frac{{Y}_{ch} - {y}_{s}}{{X}_{ch} - {x}_{s}} \\ {b}_{Mch} = {y}_{s} - {m}_{Mch} \cdot {x}_{s}\end{matrix}$$

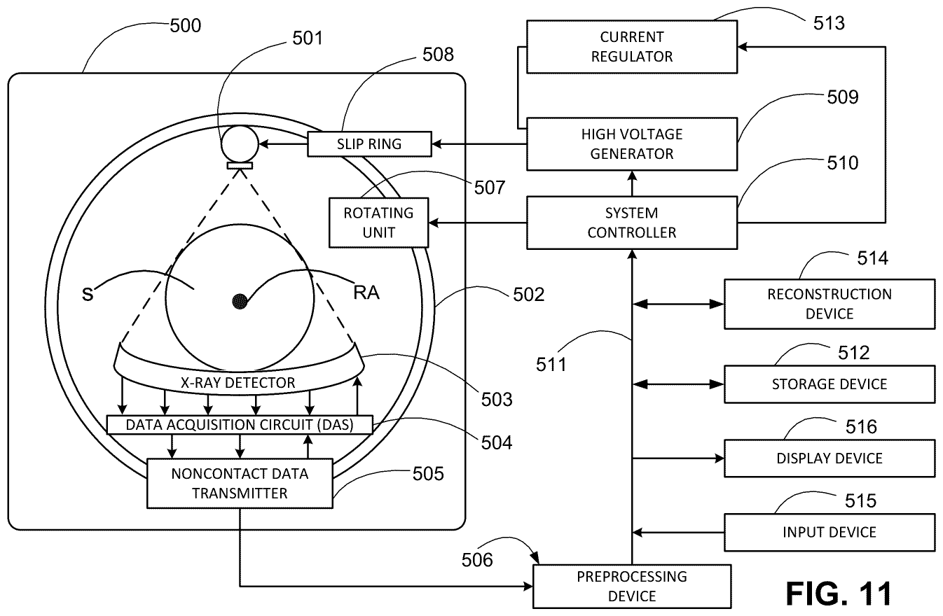

FIG. 11 illustrates an implementation of the radiography gantry included in a CT apparatus or scanner. As shown in FIG. 11, a radiography gantry 500 is illustrated from a side view and further includes an X-ray tube 501, an annular frame 502, and a multi-row or two-dimensional-array-type X-ray detector 503. The X-ray tube 501 and X-ray detector 503 are diametrically mounted across an object OBJ on the annular frame 502, which is rotatably supported around a rotation axis RA. A rotating unit 507 rotates the annular frame 502 at a high speed, such as 0.4 sec/rotation, while the object OBJ is being moved along the axis RA into or out of the illustrated page.

The first embodiment of an X-ray computed tomography (CT) apparatus according to the present inventions will be described below with reference to the views of the accompanying drawing. Note that X-ray CT apparatuses include various types of apparatuses, e.g., a rotate/rotate-type apparatus in which an X-ray tube and X-ray detector rotate together around an object to be examined, and a stationary/rotate-type apparatus in which many detection elements are arrayed in the form of a ring or plane, and only an X-ray tube rotates around an object to be examined. The present inventions can be applied to either type. In this case, the rotate/rotate type, which is currently the mainstream, will be exemplified.

The multi-slice X-ray CT apparatus further includes a high voltage generator 509 that generates a tube voltage applied to the X-ray tube 501 through a slip ring 508 so that the X-ray tube 501 generates X-rays. The X-rays are emitted towards the object OBJ, whose cross sectional area is represented by a circle. For example, the X-ray tube 501 having an average X-ray energy during a first scan that is less than an average X-ray energy during a second scan. Thus, two or more scans can be obtained corresponding to different X-ray energies. The X-ray detector 503 is located at an opposite side from the X-ray tube 501 across the object OBJ for detecting the emitted X-rays that have transmitted through the object OBJ. The X-ray detector 503 further includes individual detector elements or units.

The CT apparatus further includes other devices for processing the detected signals from X-ray detector 503. A data acquisition circuit or a Data Acquisition System (DAS) 504 converts a signal output from the X-ray detector 503 for each channel into a voltage signal, amplifies the signal, and further converts the signal into a digital signal. The X-ray detector 503 and the DAS 504 are configured to handle a predetermined total number of projections per rotation (TPPR).

The above-described data is sent to a preprocessing device 506, which is housed in a console outside the radiography gantry 500 through a non-contact data transmitter 505. The preprocessing device 506 performs certain corrections, such as sensitivity correction on the raw data. A memory 512 stores the resultant data, which is also called projection data at a stage immediately before reconstruction processing. The memory 512 is connected to a system controller 510 through a data/control bus 511, together with a reconstruction device 514, input device 515, and display 516. The system controller 510 controls a current regulator 513 that limits the current to a level sufficient for driving the CT system.

The detectors are rotated and/or fixed with respect to the patient among various generations of the CT scanner systems. In one implementation, the above-described CT system can be an example of a combined third-generation geometry and fourth-generation geometry system. In the third-generation system, the X-ray tube 501 and the X-ray detector 503 are diametrically mounted on the annular frame 502 and are rotated around the object OBJ as the annular frame 502 is rotated about the rotation axis RA. In the fourth-generation geometry system, the detectors are fixedly placed around the patient and an X-ray tube rotates around the patient. In an alternative embodiment, the radiography gantry 500 has multiple detectors arranged on the annular frame 502, which is supported by a C-arm and a stand.

The memory 512 can store the measurement value representative of the irradiance of the X-rays at the X-ray detector unit 503. Further, the memory 512 can store a dedicated program for executing various steps of the methods described herein.

The reconstruction device 514 can execute various steps of the methods described herein. Further, reconstruction device 514 can execute pre-reconstruction processing image processing such as volume rendering processing and image difference processing as needed.

The pre-reconstruction processing of the projection data performed by the preprocessing device 506 can include correcting for detector calibrations, detector nonlinearities, and polar effects, for example. Further, the pre-reconstruction processing can include various steps of the methods described herein.

Post-reconstruction processing performed by the reconstruction device 514 can include filtering and smoothing the image, volume rendering processing, and image difference processing as needed. The image reconstruction process can implement various of the steps of the methods described herein in addition to various CT image reconstruction methods. The reconstruction device 514 can use the memory to store, e.g., projection data, reconstructed images, calibration data and parameters, and computer programs.

The reconstruction device 514 can include a CPU (processing circuitry) that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory 512 can be non-volatile, such as ROM, EPROM, EEPROM or FLASH memory. The memory 512 can also be volatile, such as static or dynamic RAM, and a processor, such as a microcontroller or microprocessor, can be provided to manage the electronic memory as well as the interaction between the FPGA or CPLD and the memory.

Alternatively, the CPU in the reconstruction device 514 can execute a computer program including a set of computer-readable instructions that perform the functions described herein, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xenon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

In one implementation, the reconstructed images can be displayed on a display 516. The display 516 can be an LCD display, CRT display, plasma display, OLED, LED or any other display known in the art.

The memory 512 can be a hard disk drive, CD-ROM drive, DVD drive, FLASH drive, RAM, ROM, or any other electronic storage known in the art.

Embodiments of the radiation detector apparatus data and the functional operations described in this specification can be implemented by digital electronic circuitry, in tangibly embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of data processing apparatus, such as a networked device or server, a user devices, and the like. The computer storage medium can be a machine-readable storage device, a machine-readable storage substrate, a random or serial access memory device, or a combination of one or more of them.

The term “data processing apparatus” refers to data processing hardware and may encompass all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, Subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

According to an embodiment, the processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA an ASIC.

Computers suitable for the execution of a computer program include, by way of example, general or special purpose microprocessors or both, or any other kind of central processing unit. Generally, a CPU will receive instructions and data from a read-only memory or a random access memory or both. Elements of a computer are a CPU for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

In another embodiment, the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

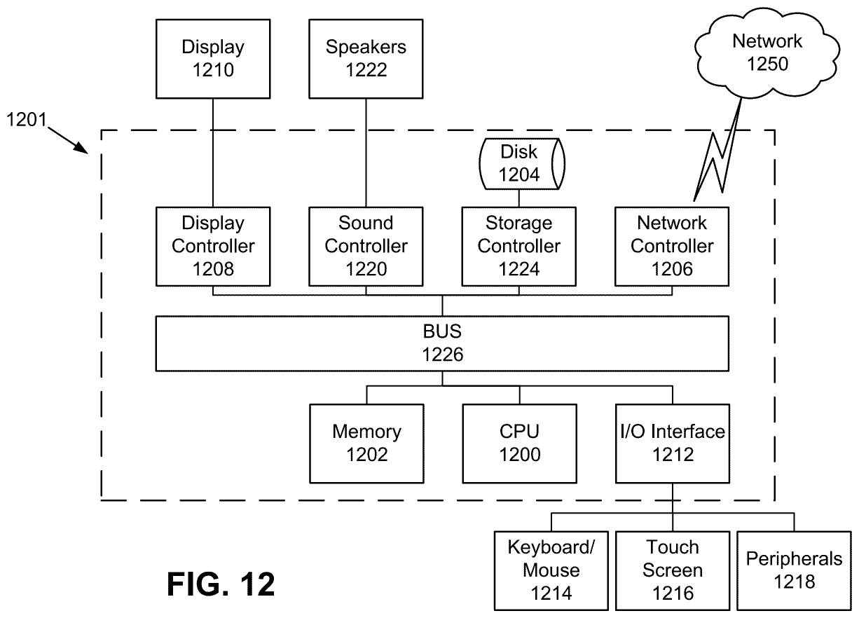

FIG. 12 illustrate a hardware description of a device 1201 according to an exemplary embodiment. In FIG. 12, the device 1201, which can be any of the above described devices, including the server and the user device, includes processing circuitry. The processing circuitry includes one or more of the elements discussed next with reference to FIG. 12. The process data and instructions may be stored in memory 1202. These processes and instructions may also be stored on a storage medium disk 1204 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 1201 communicates, such as a server or computer.

Further, the claimed advancements may be provided as a utility application, background daemon, or component of an operating system, or combination thereof, executing in conjunction with CPU 1200 and an operating system such as Microsoft Windows, UNIX, Solaris, LINUX, Apple MAC-OS and other systems known to those skilled in the art.

The hardware elements in order to achieve the device 1201 may be realized by various circuitry elements, known to those skilled in the art. For example, CPU 1200 may be a Xenon or Core processor from Intel of America or an Opteron processor from AMD of America, or may be other processor types that would be recognized by one of ordinary skill in the art. Alternatively, the CPU 1200 may be implemented on an FPGA, ASIC, PLD or using discrete logic circuits, as one of ordinary skill in the art would recognize. Further, CPU 1200 may be implemented as multiple processors cooperatively working in parallel to perform the instructions of the processes described above.

The device 1201 in FIG. 12 also includes a network controller 1206, such as an Intel Ethernet PRO network interface card from Intel Corporation of America, for interfacing with network 1250. and to communicate with the other devices. As can be appreciated, the network 1250 can be a public network, such as the Internet, or a private network such as an LAN or WAN network, or any combination thereof and can also include PSTN or ISDN sub-networks. The network 1250 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device 1201 further includes a display controller 1208, such as a NVIDIA GeForce GTX or Quadro graphics adaptor from NVIDIA Corporation of America for interfacing with display 1210, such as an LCD monitor. A general purpose I/O interface 1212 interfaces with a keyboard and/or mouse 1214 as well as a touch screen panel 1216 on or separate from display 1210. General purpose I/O interface also connects to a variety of peripherals 1218 including printers and scanners.

The general-purpose storage controller 1224 connects the storage medium disk 1204 with communication bus 1226, which may be an ISA, EISA, VESA, PCI, or similar, for interconnecting all of the components of the device 1201. A description of the general features and functionality of the display 1210, keyboard and/or mouse 1214, as well as the display controller 1208, storage controller 1224, network controller 1206, sound controller 1220, and general purpose I/O interface 1212 is omitted herein for brevity as these features are known.

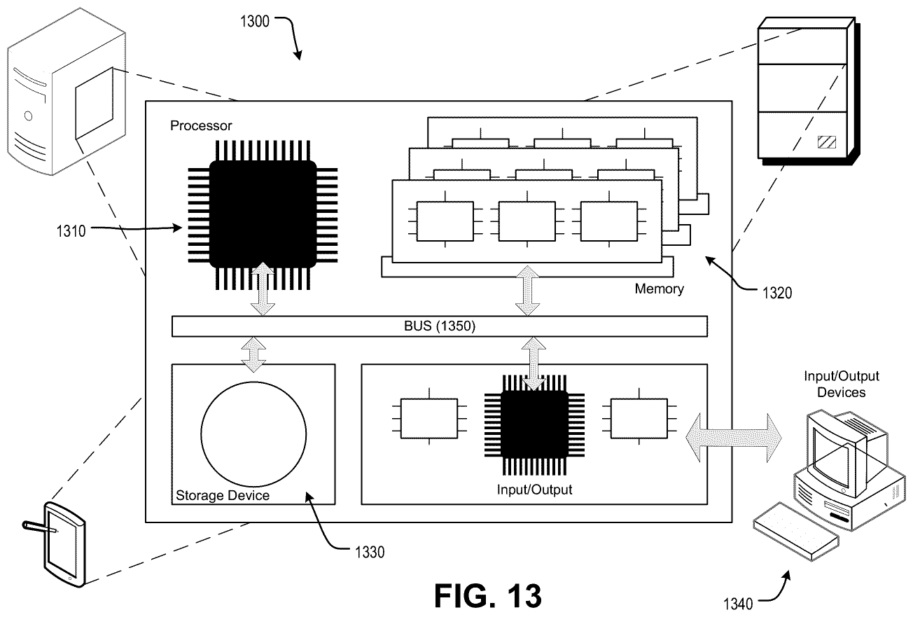

FIG. 13 illustrates an example of a type of computer. The computer 1300 can be used for the operations described in association with any of the computer-implement methods described previously, according to one implementation. For example, the computer 1300 can be an example of a networked device such as the server including processing circuitry, as discussed herein. The processing circuitry includes one or more of the elements discussed next with reference to FIG. 13. In FIG. 13, the computer 1300 includes a processor 1310, a memory 1320, a storage device 1330, and an input/output device 1340. Each of the components 1310, 1320, 1330, and 1340 are interconnected using a system bus 1350. The processor 1310 is capable of processing instructions for execution within the system 1300. In one implementation, the processor 1310 is a single-threaded processor. In another implementation, the processor 1310 is a multi-threaded processor. The processor 1310 is capable of processing instructions stored in the memory 1320 or on the storage device 1330 to display graphical information for a user interface on the input/output device 1340.

The memory 1320 stores information within the computer 1300. In one implementation, the memory 1320 is a computer-readable medium. In one implementation, the memory 1320 is a volatile memory unit. In another implementation, the memory 1320 is a non-volatile memory unit.

The storage device 1330 is capable of providing mass storage for the computer 1300. In one implementation, the storage device 1330 is a computer-readable medium. In various different implementations, the storage device 1330 may be a floppy disk device, a hard disk device, an optical disk device, or a tape device.

The input/output device 1340 provides input/output operations for the computer 1300. In one implementation, the input/output device 1340 includes a keyboard and/or pointing device. In another implementation, the input/output device 1340 includes a display unit for displaying graphical user interfaces.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous. Obviously, numerous modifications and variations of the embodiments presented herein are possible in light of the above teachings. It is therefore to be understood that within the scope of the claims, the disclosure may be practiced otherwise than as specifically described herein.

1. An apparatus, comprising:
a radiation detector including a plurality of pixels;
an anti-scatter grid (ASG) arranged on an incident side of the radiation detector, wherein a septa of the ASG is arranged over a portion of a first pixel of the plurality of pixels, but is not arranged over any portion of a second pixel of the plurality of pixels, as viewed from the incident side of the radiation detector; and
processing circuitry configured to
receive a first detection result from the first pixel;
receive a second detection result from the second pixel;
estimate a positional offset of the septa of the ASG, based on the first detection result and the second detection result.

2. The apparatus of claim 1, wherein the processing circuitry is further configured to
estimate a length of a shadow cast by the septa on the first pixel based on the first detection result and the second detection result; and
estimate the positional offset of the septa based on the estimated length of the shadow.

3. The apparatus of claim 2, wherein the processing circuitry is further configured to estimate the length of the shadow further based on a width of the first pixel, and estimate the positional offset of the septa further based on a width of the septa.

4. The apparatus of claim 1, wherein the processing circuitry is further configured to estimate the positional offset of the septa, the positional offset being one of a right offset and a left offset.

5. The apparatus of claim 1, wherein the processing circuitry is further configured to estimate the positional offset of the septa, the positional offset being one of a rotation up and a rotation down.

6. The apparatus of claim 1, wherein
the ASG comprises a plurality of septa, which are radiation absorptive members, and a plurality of radiation transmissive members, alternatively arranged in a form of slits or a matrix,
the radiation detector apparatus comprises a plurality of detector module blades (DMB),
each of the plurality of DMB includes a plurality of submodules comprising a two-dimensional array of micropixels arranged in a plurality of rows and a plurality of columns,
the plurality of columns of each of the plurality of submodules are arranged in channels consisting of three of the columns, each channel including a left microchannel, a center microchannel, and a right microchannel, and
a first septa of the ASG is disposed on a left edge of the left microchannel and a second septa of the ASG is disposed on a right edge of the right microchannel.

7. An apparatus for detecting misalignment of an anti-scattering grid (ASG) offset detection for a radiation detector apparatus, the apparatus comprising:
a radiation detector including a plurality of pixels, the plurality of pixels being arranged in a plurality of modules; and
processing circuitry configured to
acquire first counts obtained from a first air scan with an anti-scatter grid (ASG) having a plurality of septa arranged on an incident side of the radiation detector;
acquire second counts obtained from a second air scan without the ASG arranged on the radiation detector;
calculate third counts by normalizing the first counts based on the second counts;
estimate positional offsets of the plurality of septa of the ASG based on the calculated third counts;
determine whether any particular module of the plurality of modules is faulty based on the estimated positional offsets of the plurality of septa.

8. The apparatus of claim 7, wherein the processing circuitry is further configured to modify the acquired second counts based on the sizes of the plurality of pixels.

9. The apparatus of claim 7, wherein the processing circuitry is further configured to determine whether any particular module of the plurality of modules is faulty based on an average positional offset computed for a group of the plurality of septa of the ASG within the particular module.

10. An apparatus for detecting a tilt angle of an X-ray source, comprising:
a radiation detector including a plurality of pixels, the plurality of pixels being arranged in a plurality of channels;
an anti-scatter grid (ASG) arranged on an incident side of the radiation detector, wherein, for each channel of the plurality of channels, as viewed from the incident side of the radiation detector, (1) a first septa of the ASG is arranged over a portion of a first pixel of the channel, but is not arranged over any portion of a second pixel of the channel that is adjacent to the first pixel, and (2) a second septa of the ASG is arranged over a portion of a third pixel of the channel that is adjacent to the second pixel, but is not arranged over any portion of the second pixel, the first, second, and third pixels forming the channel; and
processing circuitry configured to, for each channel of the plurality of channels,
acquire a first detection result from the first pixel, a second detection result from the second pixel, and a third detection result from the third pixel, the detection results being obtained from an air scan with the ASG using the radiation source;
estimate a right offset of the first septa, based on the first detection result and the second detection result;
estimate a left offset of the second septa, based on the third detection result and the second detection result;
generate a mean right offset by averaging the estimated right offset over each of the channels, and generate a mean left offset by averaging the estimated left offset over each of the plurality of channels;
determine the tilt angle of the X-ray source based on the generated mean right offset and the generated mean left offset.

11. The apparatus of claim 10, wherein the processing circuitry is further configured to determine the tilt angle by
determining a sign of the tilt angle based on the generated mean right offset and the generated mean left offset,
selecting a predetermined lookup table based on the determined sign of the tilt angle, and
determining a value of the tilt angle based on one of the generated right mean offset and the generated left mean offset using the selected predetermined lookup table.

12. The apparatus of claim 11, wherein the processing circuitry is further configured to, in selecting the predetermined lookup table,
select a right offset lookup table when determining that the generated right mean offset is greater than the generated left mean offset, and
select a left offset lookup table when determining that the generated right mean offset is less than the generated left mean offset.

13. The apparatus of claim 11, wherein the processing circuitry is further configured to, in determining the sign of the tilt angle,
determine that the sign of the tilt angle is positive when determining that the generated right mean offset is greater than the generated left mean offset, and
determine that the sign of the tilt angle is negative when determining that the generated right mean offset is less than the generated left mean offset.

14. The apparatus of claim 12, wherein the processing circuitry is further configured to pre-calculate the right offset lookup table and the left offset lookup table by
obtaining simulated count data obtained for each tilt angle of a range of positive tilt angles and a range of negative tilt angles; and
calculating, for each tilt angle, left and right offsets for each of the plurality of channels;
calculating, for each tilt angle, an average left offset and an average right offset;
associating, for each positive tilt angle, a corresponding average right offset, to form the right offset lookup table; and
associating, for each negative tilt angle, a corresponding average left offset, to form the left offset lookup table.

15. The apparatus of claim 10, wherein the processing circuitry is further configured to determine the tilt angle of the X-ray source with respect to an isocenter and a center line of the radiation detector.

16. The apparatus of claim 10, wherein the processing circuitry is further configured to
estimate a length of a first shadow cast by the first septa on the first pixel based on the first detection result and the second detection result;
estimate the right offset of the first septa based on the estimated length of the first shadow;
estimate a length of a second shadow cast by the second septa on the third pixel based on the third detection result and the second detection result; and
estimate the left offset of the second septa based on the estimated length of the second shadow.

17. The apparatus of claim 16, wherein the processing circuitry is further configured to
estimate the length of the first shadow further based on a width of the first pixel, and estimate the right offset of the first septa further based on a width of the first septa; and
estimate the length of the second shadow further based on a width of the third pixel, and estimate the left offset of the second septa further based on a width of the second septa.
